(19) United States
(12) Patent Application Publication
(10) Pub. No.: US 2007/0010774 A1
(43) Pub. Date: Jan. 11, 2007

(54) TRACTION DEVICE WITH DIAPHRAGM AND MANIFOLD CONNECTION

(75) Inventors: Christian HUNT, McLean, VA (US); Ara BOGHOSIAN, Alexandria, VA (US); John A. SPADAVECCHIA, Red House, VA (US)

Correspondence Address: GREENBLUM & BERNSTEIN, P.L.C., 1950 ROLAND CLARKE PLACE, RESTON, VA, 20191, US

(73) Assignee: CARE REHAB & ORTHOPEDIC PRODUCTS, INC., McLean, VA (US)

(21) Appl. No.: 11/275,969

(22) Filed: Feb. 7, 2006

Related U.S. Application Data
(60) Provisional application No. 60/653,106, filed on Feb. 16, 2005.

(51) Int. Cl. A61F 5/00

(52) U.S. Cl. 602/32

(57) ABSTRACT

A device includes a stationary housing. A carriage is slidable mounted on to the housing and at least one corrugated diaphragm mechanism is housed in the housing and connectable to the carriage. The corrugated diaphragm is structured to elastically expand and retract, depending on an application of pressure, to extend and retract, respectively, the carriage relative to the stationary housing. A manifold is provided to connect the corrugated diaphragm to a pump system.

### CROSS REFERENCE TO RELATED APPLICATIONS

This application claims priority to U.S. Provisional Application Ser. No. 60/653,106, filed on Feb. 16, 2005, the contents of which are incorporated in its entirety herein. This application is also related to co-pending U.S. application Ser. No. ______, filed on ______ (Attorney Docket No. 26866) which also claims priority to U.S. Provisional Application Serial No. 60,653,106, filed on Feb. 16, 2005, the contents of which are incorporated in its entirety herein.

### FIELD OF THE INVENTION

The invention generally relates to a cervical and/or lumbar traction device, and more particularly, to a cervical and/or lumbar traction device having a diaphragm with a manifold and connection thereof.

### DISCUSSION OF BACKGROUND INFORMATION

Traction devices are used to relieve pressure on inflamed or enlarged nerves. Cervical and lumbar or spinal traction devices are the most common type of these devices. When correctly used, the traction devices can relieve pain in the neck and the spine by, for example, straightening the curvature of the spine or stretching of the spinal and cervical musculature.

Portable traction devices are now becoming very popular for in home use. These devices allow patients to perform traction therapy without leaving their homes, or expending large sums of money for a healthcare provider or physical therapist. Under the proper guidance and instruction, these portable devices are becoming ever more common, especially in today's age of rising health care costs.

Additionally, known portable lumbar traction devices, currently manufactured and sold, are cumbersome and difficult to transport. These lumbar traction devices are basically a large board of approximately 3 or more feet in length.

In one known portable lumbar traction device, two flat separate boards are assembled in order to form the platform for the device. To make such assembly, hooks extending from one flat board are aligned with holes in the other flat board. This is accomplished, most typically, by lifting and aligning the board with the hooks and then inserting the hooks into the holes. During this assembly, a piston rod of a pneumatic device remains attached to one board and the cylinder housing of the pneumatic cylinder remains attached to the other board, making for a very awkward assembly. To disassemble the traction device, the hooks must be removed from the holes, which is an awkward process, especially in view of the piston rods and cylinders remaining attached between the separated boards.

### SUMMARY OF THE INVENTION

In a first aspect of the invention, a device includes a frame and a carriage slidable mounted on the frame. At least one diaphragm mechanism is connectable to the carriage. The diaphragm is structured to elastically expand and retract, depending on an application of pressure, in order to extend and retract, respectively, the carriage. A manifold is coupled between the at least one diaphragm and a pump system. The manifold includes at least one port coupled to a neck of the at least one diaphragm and a neck communicating with the pump system.

In another aspect of the invention, the device includes a frame for accommodating at least one corrugated diaphragm device. The at least one corrugated diaphragm device has a retractable spring force that, upon release of pressure within the at least one corrugated diaphragm device, the at least one corrugated diaphragm device retracts and moves the carriage towards an initial position. A manifold is connectable between the least one corrugated diaphragm device and a pneumatic system. The manifold includes at least one port and at least one neck. The at least one port is connectable to a neck of the least one corrugated diaphragm device and the at least one neck is connectable to a hose of the pneumatic system.

In another aspect of the invention, A manifold device includes a body having at least one port and one neck having a channel therein in fluid communication with the at least one port. The neck includes a projection. An adapter is configured to be insertabe into the at least one port. The adapter comprises a varied cross section with a major diameter of at least one portion of the varied cross section being larger than an inner diameter of the at least one port at least in one location.

### BRIEF DESCRIPTION OF THE DRAWINGS

The present invention is further described in the detailed description which follows, wherein:

FIG. 1 shows a top view of a cervical traction device in accordance with the invention;

FIGS. 2a and 2b show side views of the cervical traction device in an extended and retracted position, respectively, in accordance with the invention;

FIG. 3 shows a cut-away view of the cervical traction device in accordance of the invention;

FIG. 4 shows an exploded view of the cervical traction device in accordance with the invention;

FIG. 4a and FIG. 4b (cross section) show a connection mechanism in accordance with the invention;

FIG. 5 shows a bottom view (with a bottom housing removed) of the cervical traction device in accordance with the invention;

FIG. 5a shows a top view (with cushion wedges removed) of the cervical traction device in accordance with the invention;

FIG. 6 shows a lumbar traction device in accordance with the invention;

FIG. 7 shows an exploded view of the lumbar traction device in accordance of the invention

FIG. 8 shows a perspective view of a corrugated diaphragm and manifold, in accordance with the invention;

FIG. 9 shows a cutaway of the corrugated diaphragm and manifold along line 9-9 of FIG. 8, in accordance with the invention;

FIG. 10 shows a perspective view of a barb adapter in accordance with the invention;

FIG. 11 shows another embodiment of the corrugated diaphragm and manifold, in accordance with the invention;

FIG. 12 shows a manifold with pressure release valve in accordance with the invention; and

FIG. 13 shows a cross section of the manifold along line 12-12 of FIG. 12.

### DETAILED DESCRIPTION OF EMBODIMENTS OF THE INVENTION

The invention is directed to a cervical and/or a lumbar traction device. The traction device includes a corrugated diaphragm which is structured and adapted to move a carriage in a linear fashion. The diaphragm is connected to a pump via a manifold and connection thereof. The diaphragm includes a certain spring force which is capable, upon the release of pressure therein, to move the carriage towards its original position and, in embodiments, substantially to its original position. In addition to its own spring force, a roll spring or other spring or resilient type mechanism may be used in combination with the diaphragm, to retract the carriage towards its original position. The diaphragm is devoid of gaskets and other moving parts. The cervical and/or lumbar traction device, using the diaphragm and related components, is lighter and includes fewer parts than devices using pneumatic cylinders.

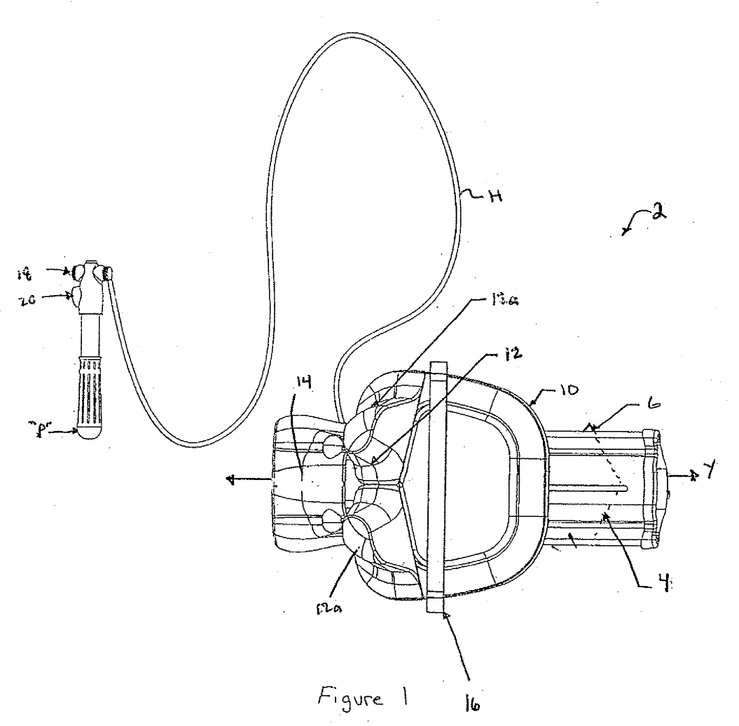

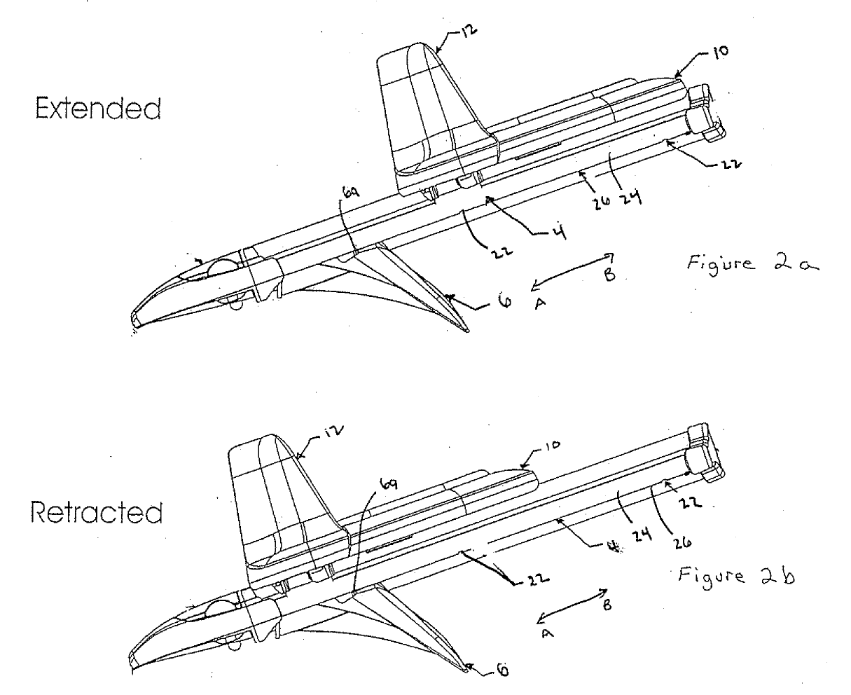

FIG. 1 shows an embodiment of the cervical traction device. The cervical traction device is generally denoted as reference numeral 2. The traction device 2 includes a stationary housing (e.g., frame) 4 having a moveable stand 6 which is structured and adapted so that several angles can be achieved relative to a flat surface, e.g., a floor or other surface where a user may use the device 2. The cervical traction device 2 further includes a movable headrest 10 having a occiput wedge system 12, which is mounted to a sliding carriage 14. A strap or other restraining device 16 for restraining a users head is attached to the movable head-rest system 10.

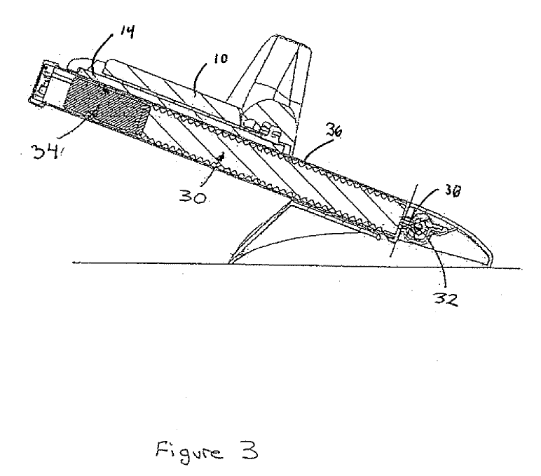

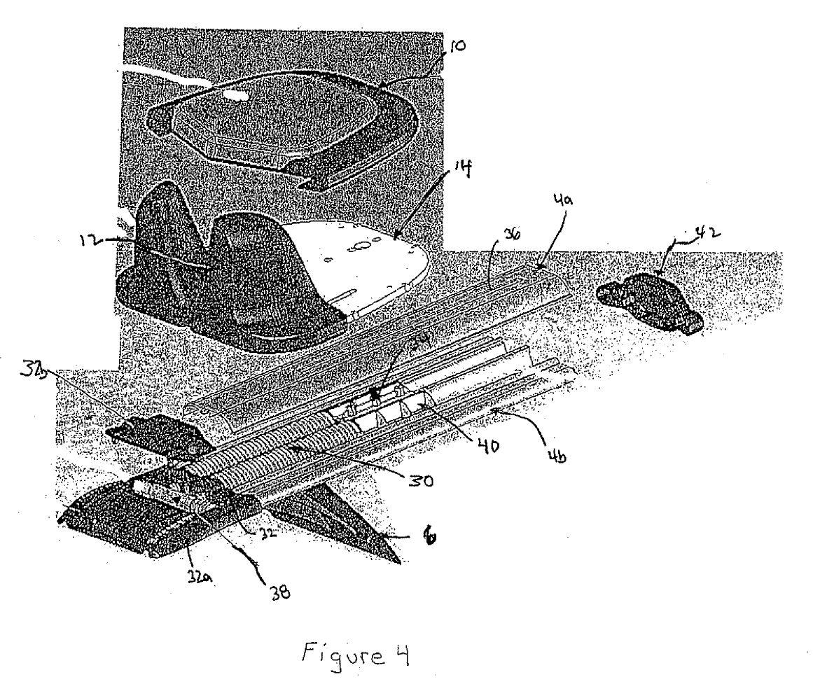

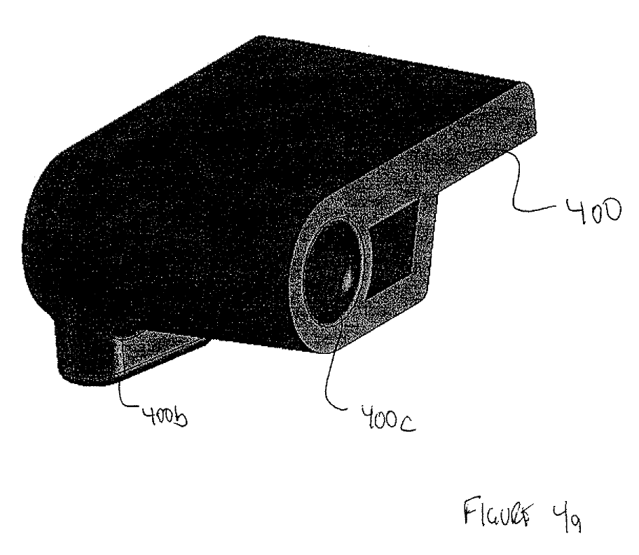

The occiput wedge system 12 includes separate wedges 12a that may have a concave engaging surface. The occiput wedge system 12, with the head-rest 10, may be slidably movable along a longitudinal axis “Y” of the housing 4 by a corrugated diaphragm type device (shown more clearly in FIG. 3). The occiput wedge system 12 is designed to apply a therapeutic traction force to the occipital areas on a patent's head, while the housing 4 remains stationary. This is accomplished, in one aspect of the invention, by pressure being applied by a pump P via a hose H, to the diaphragm type device. Thus, upon pressurization, the corrugated diaphragm type device will move or slide the carriage 14 and thus the head-rest 10 and occiput wedge system 12. The pump P may have a manual pressure relief mechanism 18, as well as a gauge 20 to show a force created on the diaphragm type device.

FIGS. 2a and 2b show the cervical traction device 1 positioned at an angle of, for example, 10 degrees relative to a flat surface, e.g., a floor or other surface where the user may use the device 2. It should be understood that other angles of adjustment, e.g., 15 degrees, 20 degrees, etc. with relation to the supporting surface, are also contemplated by the invention, and that the angles shown herein are provided for illustrative purposes. In the examples of FIGS. 2a and 2b, the adjustment can be accomplished via movement of the moveable stand 6 in either direction “A” or “B”, along the longitudinal axis of the device 2.

Still referring to FIGS. 2a and 2b, the housing 4 includes a guide 24 which has turned ends that form rails 26. The rails 26 are structured to accommodate the stand 6. In one embodiment, the rails 26 include a plurality of notches 22 for locking or positioning of the stand 6 at a predetermined position. The notches 22 may also be apertures or holes. In embodiments, the stand 6 may be locked into place by use of a spring arm, hook or other type of mechanism 6a that engages the notches 22 positioned along the stand 6 of the housing 4.

As discussed in greater detail below, in FIG. 2a, the traction device is in an extended position; whereas, in FIG. 2b, the traction device is in a retracted position. The extended position is achievable by pressurizing the diaphragm shown in FIG. 3. The retracted position is achievable, at least in part, by a return spring force of the diaphragm. That is, upon release of pressure, the spring force of the diaphragm is capable of moving the carriage towards its original position and, in embodiments, substantially to its original position. In addition to its own spring force, a roll spring or other spring or resilient type mechanism may be used in combination with the diaphragm, to retract the carriage towards its original position.

FIG. 3 shows a cutaway view of the traction device in accordance with the invention. The traction device 2 includes a corrugated diaphragm type device 30 that is mounted within or mounted to the housing 4. In embodiments, the corrugated diaphragm type device 30 is mounted to a support structure 32 of the housing 4 and the movable carriage 14. In one implementation, at least one boss 34 extends from the moveable carriage 14, via a slot 36 extending substantially along a length of the housing 4, into an interior space of the housing 4, which is connected to a mounting carriage 35. In this embodiment, the corrugated diaphragm type device 30 is attached to the at least one boss 34 via a mounting carriage 35 adapted and configured to slide within the housing 4 and preferably a profile 35a of the housing 4 that is configured to house the corrugated diaphragm type device 30, In alternate embodiments, the corrugated diaphragm type device 30 is mounted or attached to a mounting carriage which has at least one the extending boss adapted and configured to slide within a guide of the housing 4 and connectable to the sliding carriage 14. Upon pressurization of the corrugated diaphragm type device 30, the corrugated diaphragm type device 30 will move the carriage 14 to apply a traction force.

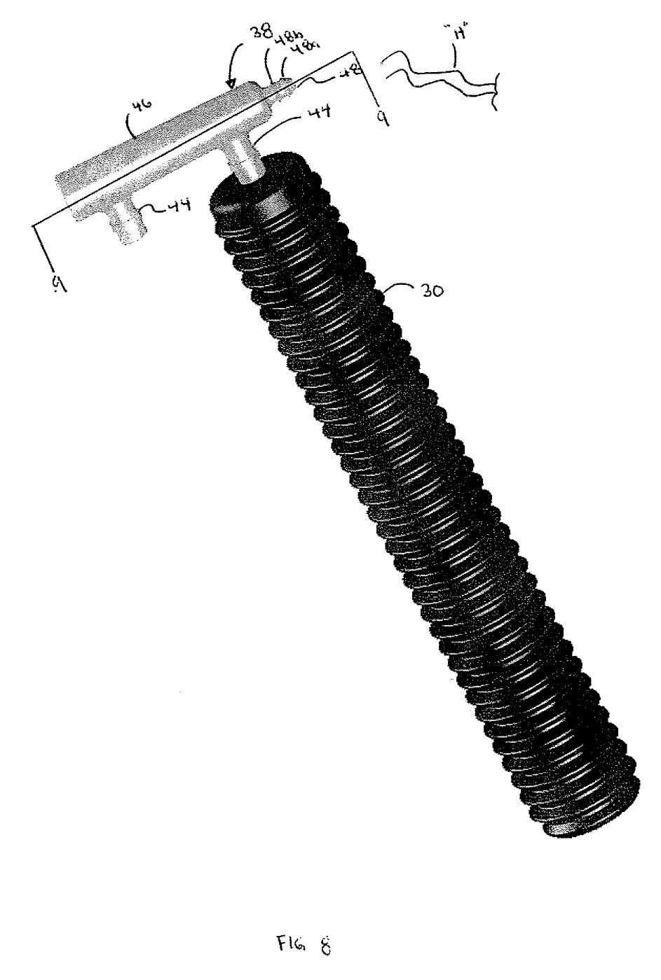

The corrugated diaphragm type device 30 is connected to an air inlet or manifold 38 provided at an end of the housing 4, proximate to the support structure 32. This connection should preferably minimize any leakage of air, at this junction. The manifold 38 is connected to one or more of the diaphragm type devices 30 and is structured to allow pressurized air from the pump “P” to pass into the corrugated diaphragm type device 30. The manifold and connection thereof is discussed in more detail with reference to FIGS. 8-10.

Still referring to FIG. 3, the corrugated diaphragm type device 30 acts as a spring, e.g., has its own spring force, to retract the carriage in both a cervical and lumbar traction system from an expanded position. In one exemplary illustration, the corrugated diaphragm type device 30 can be expanded to approximately 10.5 inches in the expanded position, and in one embodiment may have a starting position of about 6.5 inches in length (with an original neutral length of approximately 8 inches). It is also contemplated that other starting and expanded positions are provided by the invention. In one implementation, a stroke length of the corrugated diaphragm type device 30 is approximately 4 inches; although other stroke lengths are also contemplated by the invention.

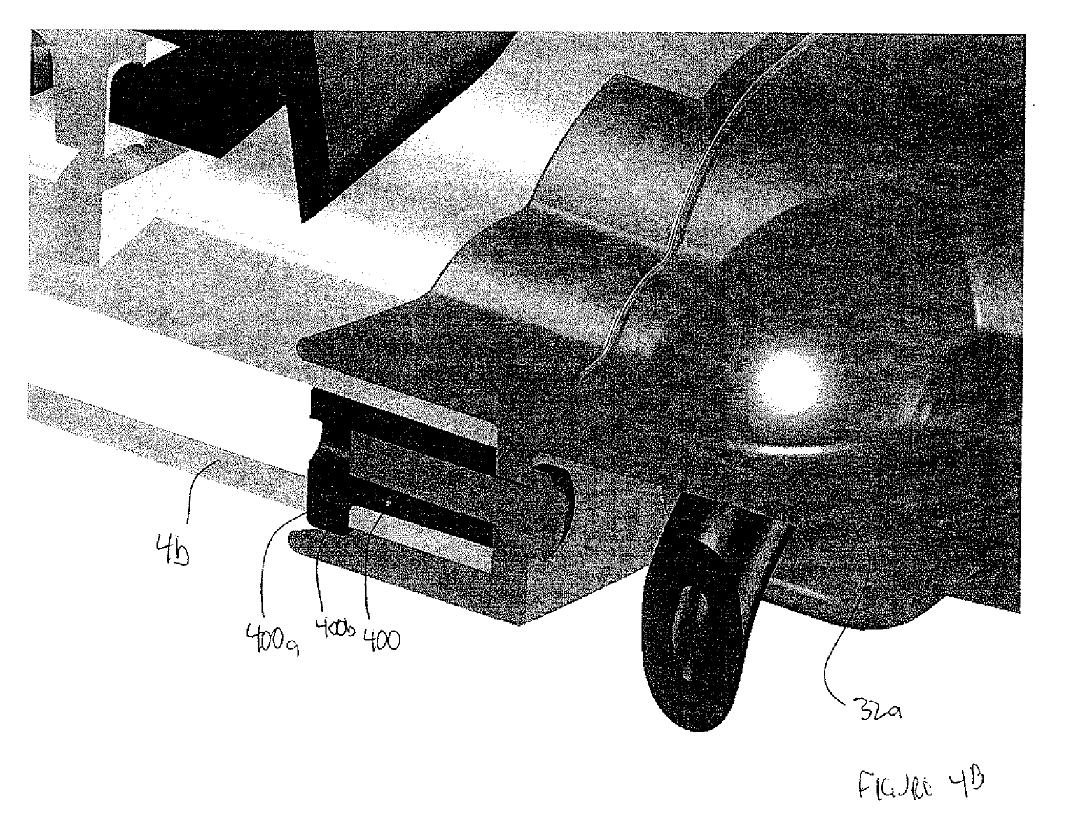

FIG. 4 shows an exploded view of the traction device in accordance with the invention. The traction device includes, for example, the headrest 10 and occiput wedge system 12 connected to the carriage 14. The carriage 14, in turn, is connected to the at least one boss 34 which extends through the slot 36 along the housing 4 and more specifically a cover plate 4a of the housing 4. The housing 4 further includes a lower frame portion 4b, which includes an interior portion 40 for housing the corrugated diaphragm type device 30.

The housing 4 further includes the support structure 32, which may include an extending portion 32a for resting on a supporting surface. A cover 32b may be used, in conjunction with the extending portion 32a, to form an interior space, which accommodates the manifold 38. An end cap 42 may be connected to the housing 4, opposing the extending portion 32a. Thus, the combination of the end cap 42, top plate 4a, bottom frame 4b and support structure forms an interior housing for the corrugated diaphragm type device 30.

The extending portion 32a may be held in place by a nut 400 having a projection 400b, as shown in FIGS. 4a and 4b. In this implementation, the nut will be bolted via the hole 400c and the projection 400b will communicate with a slot 400a in the frame portion to prevent rotation of the extending portion 32a and to ensure a connection therebetween Alternatively, the extending portion 32a may be connected or mated with the frame by any known, conventional manner such as a screw, nut and bolt system, etc.

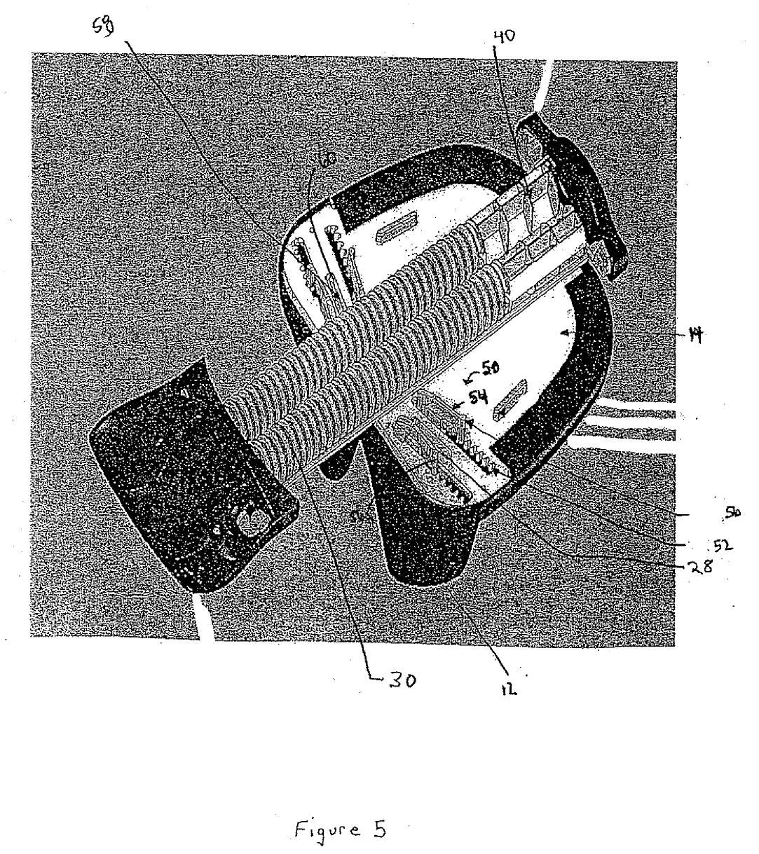

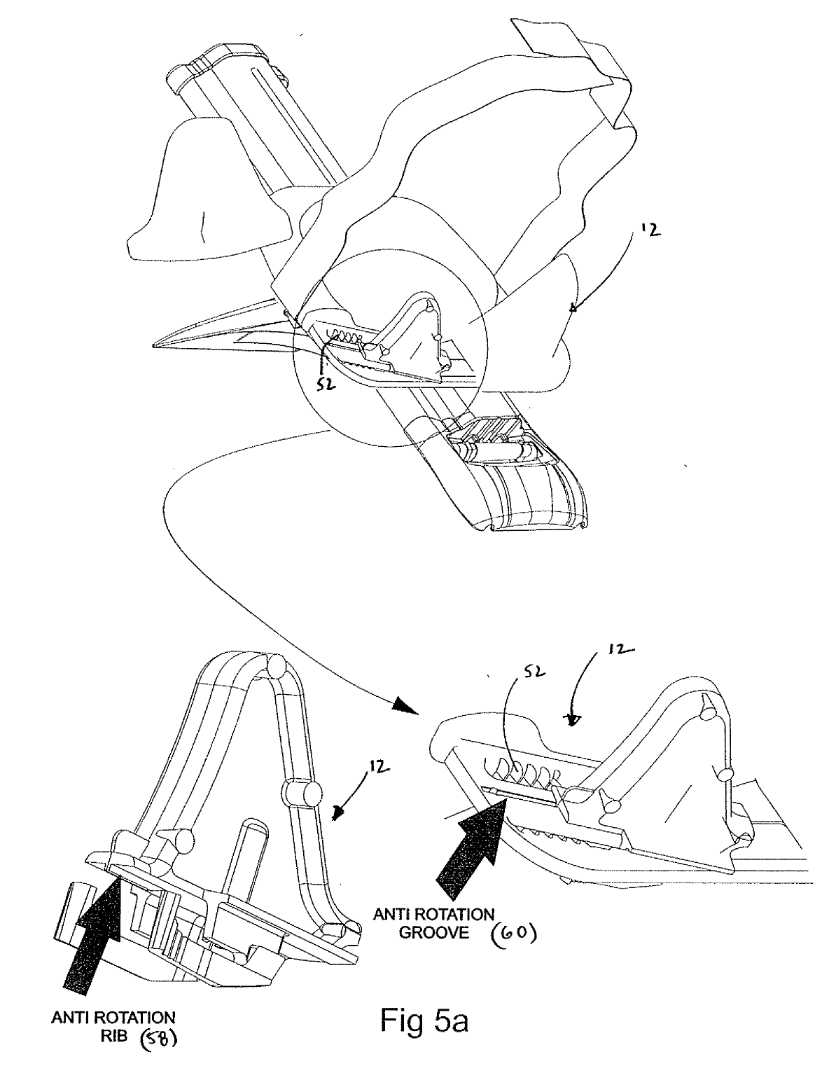

FIG. 5 shows a bottom cutaway view of the traction device 2 in accordance with the invention. In this example, the wedges 12 are designed to move in a lateral direction, e.g., do not rotate. In this embodiment, the wedges 12 can be adjustable by a pinch spring arm mechanism 50. The pinch spring arm mechanism 50 includes a ratchet type guide 52 in/on the moveable carriage 14. The pinch spring arm mechanism 50 further includes spring arms 54 which may engage the ratchet guide 52 at different positions via opposing protruding portions 56. The opposing protruding portions 56 have tabs 56a so that the user can squeeze them together to disengage from a ratchet portion of the ratchet guide 52, and then move the wedges in an inward position or an outward position. The movement of the wedges may be in a somewhat linear manner across the carriage 14.

Still referring to FIG. 5 and additionally FIG. 5a, the wedge system 12 may also include an anti-rotation rib 58 and corresponding slot or groove 60 for preventing rotation of the wedge 12. The slot 60 can be part of the carriage 14 and is, in one implementation, proximate to the ratchet guide 52. The rib 58 communicates with the slot 60 while allowing the wedges 12 to slide in and out.

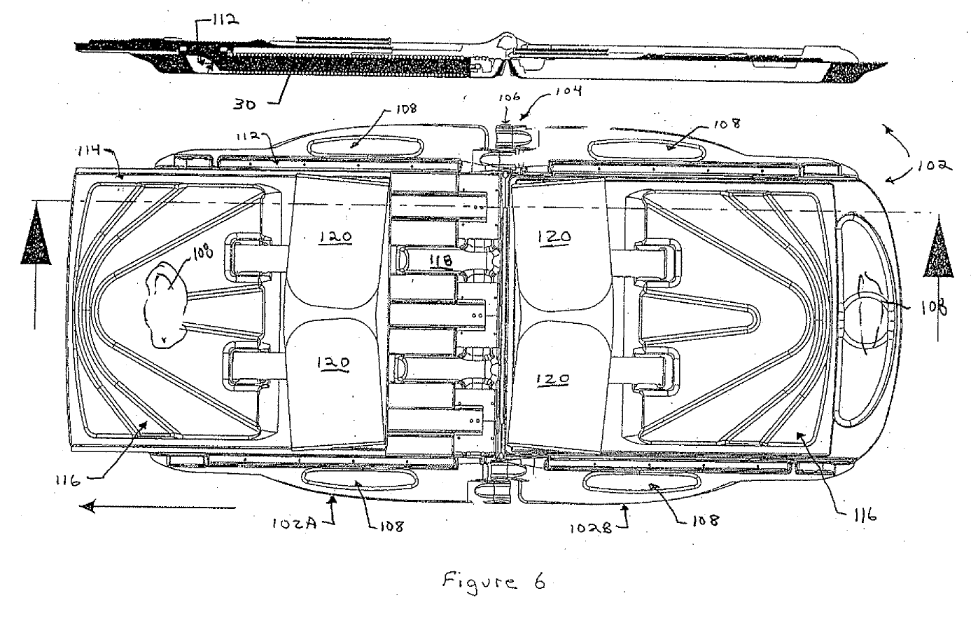

FIG. 6 shows an embodiment of a lumbar traction device 100 in accordance with the invention. Similar to the traction device shown and described with reference to FIGS. 1-5, a corrugated diaphragm type device is used to apply a traction type force. In the lumbar traction device 100, three corrugated diaphragm type devices may also be used to provide the traction force; although it is contemplated that more or less corrugated diaphragm type devices may be used in accordance with the invention. Integrally molded frame members 102A and 102B are hingedly attached via a hinge type mechanism 104 (which may be an alternating type hinge mechanism having at least four parts) to form a portable, foldable device. In the closed position, for example, the frame members 102A and 102B face one another; whereas, in the open state, the frame members 102A and 102B extend along a longitudinal axis in a single plane. Thus, the design of the traction device of FIG. 6 allows for the easy closing and opening of the lumbar traction device 100 without the requirement for aligning any parts, lifting any of the parts or separating any of the parts. The lumbar traction device 100 also includes integrally formed handles 108, for example, molded on sides or the ends thereof.

Still referring to FIG. 6, the frame members 102A and 102B are designed to house or mount thereon many of the components of the lumbar traction device 100, such as the corrugated diaphragm type device 30. By way of illustration, the lumbar traction device 100 may include guides 112 positioned on opposing sides of the frame member 102A and/or frame member 102B. The guides 112 are designed to seat thereon a slidable lumbar carriage 114. The lumbar carriage 114 is slidably moveable along the longitudinal axis of the frame 102A and 102B to provide a traction force such that, upon pressurization or release thereof, the corrugated diaphragm type device moves the slidable lumbar carriage 114 in directions “A” and “B”. In embodiments, the lumbar carriage 114 includes a support pad 116 for the comfort of a user. A pair of belts or other restraining device 120 can be mounted to the support pad 116 and/or the frame member(s). The slidable lumbar carriage 114 may also be seated within a recessed portion 118 of the frame.

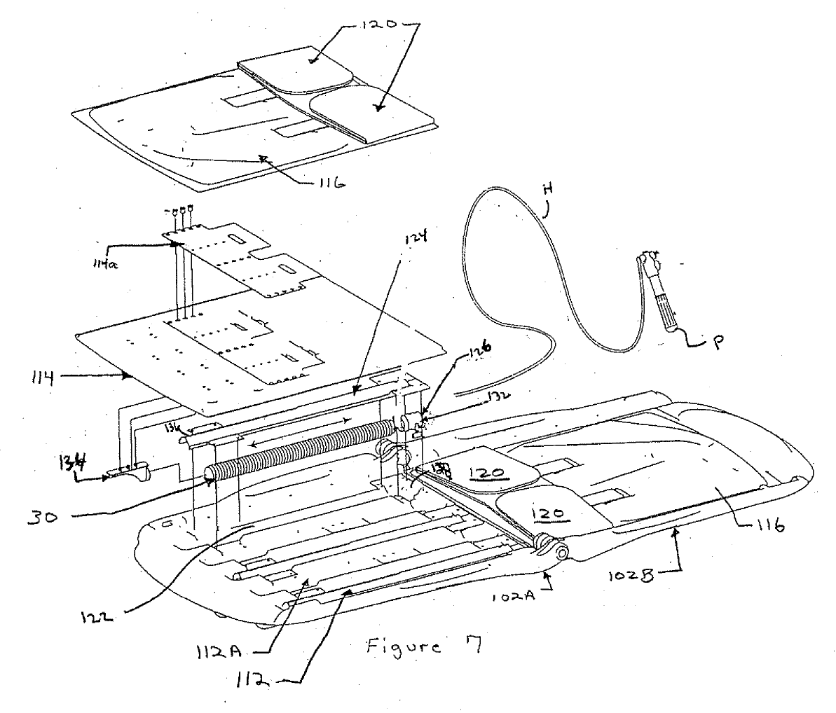

Referring to FIG. 7, the slidable lumbar carriage 114 is connected to the corrugated diaphragm type device 30 in order to provide a traction force. Much like discussed above, the corrugated diaphragm type device 30 is mounted within a housing or recess 122 formed by the frame member and a cover plate 124. In embodiments, the lumbar traction device may include two or three corrugated diaphragm type devices 30, corresponding to the number of recesses and/or the required application of traction force.

The corrugated diaphragm type device 30 is connected to the manifold 126 which, in turn, is housed inside support structure 132, which, in turn, is mounted to support structure 128. The manifold 126 includes an air intake/exhaust 132 connected to a hose “H” of the hand pump “P”. The corrugated diaphragm type device 30 is also connected to the slidable lumbar carriage 114 via at least one boss 134. In one implementation, the at least one boss 134 extends from the slidable lumbar carriage 114, via s slot or cutout 136 extending substantially along a length of the plate 124. In this embodiment, the corrugated diaphragm type device 30 is attached to the at least one boss 134 and, upon pressurization of the corrugated diaphragm type device(s) 30, will move the slidable lumbar carriage 114 to apply a traction force. The support pad 116 may be mounted to the slidable lumbar carriage 114 via a plate 114a (and fasteners, e.g., screws, rivets, etc.)

Still referring to FIG. 7, the corrugated diaphragm type device 30 acts as a spring, e.g., has its own spring force, to retract the carriage in both a cervical and lumbar traction system from an expanded position. In addition to its own spring force, a roll spring or other spring or resilient type mechanism may be used in combination with the diaphragm, to retract the carriage towards its original position which is represented schematically with the diaphragm. In one exemplary illustration, the corrugated diaphragm type device 30 can be expanded to approximately 10.5 inches in the expanded position, and in one embodiment may have a starting position of about 6.5 inches in length. It is also contemplated that other starting and expanded positions are provided by the invention. In one implementation, a stroke length of the corrugated diaphragm type device 30 is approximately 4 inches; although other stroke lengths are also contemplated by the invention.

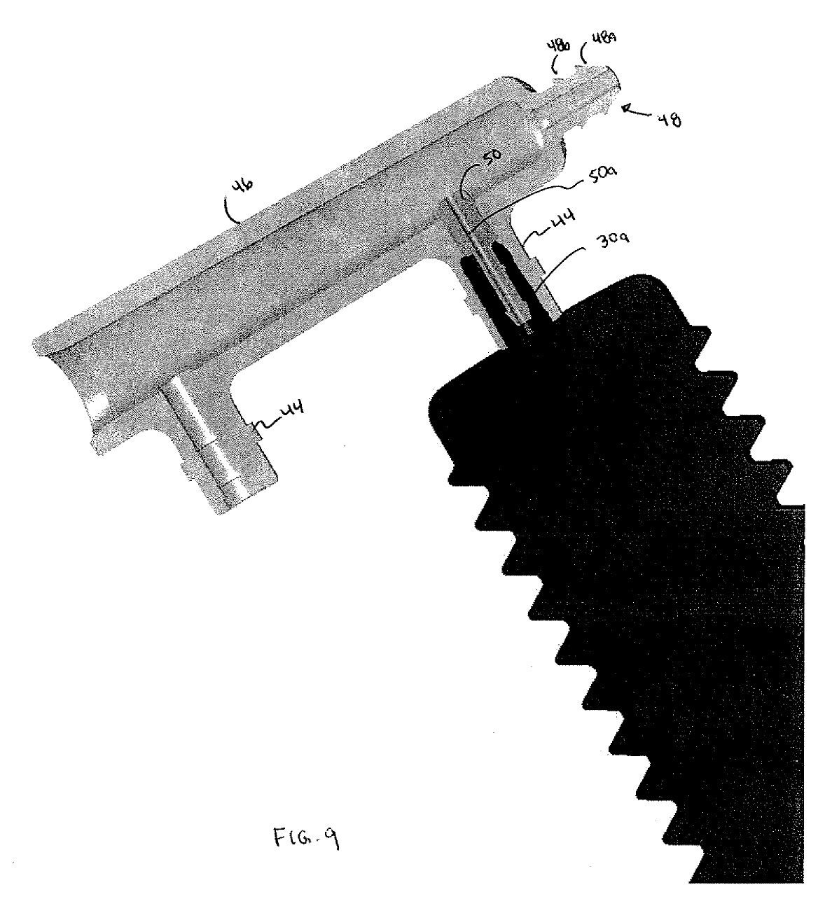

FIGS. 8 and 9 show a corrugated diaphragm and manifold system. For purposes of this discussion, reference will be made to the manifold 38; although, it should be understood that the discussion herein is equally applicable to the manifold 126. In embodiments, two corrugated diaphragms 30 (with only one shown for illustrative purposes) are coupled to or mated with the manifold 38 via ports (e.g., hollow necks) 44 extending along a length of a body 46. In other embodiments, the manifold 38 may include more or less than two ports in order to couple or mate more or less than two corrugated diaphragms, depending on the desired or required forces needed to move and/or retract the carriage of the traction device. For example, a spring force of one corrugated diaphragm may be designed to move the traction device to the retracted position.

In embodiments, the ports 44 are in fluid communication with the hollow body 46 which, in turn, is in fluid communication with a neck 48 positioned at one end of the manifold 38; although, it should be understood that the neck 48 may be placed at any location along the hollow body 46. The neck 48 includes one or more outward extending barbs 48a, 48b, e.g., conically shaped protrusions which facilitate the engagement of the hose “H” to the manifold 38.

In embodiments, the hose “H” is mated to the neck 48 and held securely in place via the one or more barbs 48a, 48b; that is, the hose “H” is placed about the neck 48 and is securely attached thereto by the use of the barbs 48a, 48b. In embodiments, the barb 48a includes a tapered end in order to facilitate the placement of the hose “H” over the neck 48. In further embodiments, an outermost diameter of at least one of the barbs is slightly larger than an inner circumference of the hose “H” to minimize leakage between the hose “H” and the manifold 38. Additionally, as should be understood by those of skill in the art, the direction of the barbs, facing toward the body 46, facilitates easy installation of the hose “H” while ensuring that the hose “H” will not disengage from the neck 48. In embodiments, the hose “H” is made of a flexible and/or resilient material such that it slightly deforms as it is moved over the barbs of the neck 48, thus ensuring a snug, tight fit between the hose “H” and the neck 48.

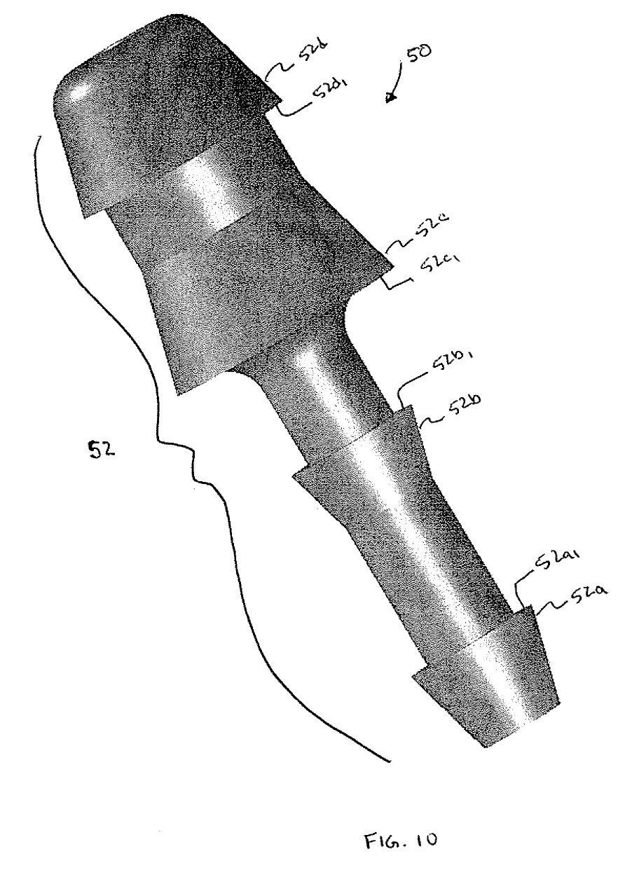

Still referring to FIGS. 9 and 10, in one embodiment, the corrugated diaphragm 30 can be secured to the port 44 via a glueless mechanism (e.g., a barb adapter 50). For example, as discussed in more detail below, the barb adapter 50 provides an outward force to ensure the neck 30a of the corrugated diaphragm 30 remains engaged to the barb adapter. The connection between the major diameter of the barb adaptor and the inner diameter of the neck of the diaphragm and more specifically a barb feature on the diaphragm minimizes air leakage.

The connection mechanism 50 (e.g., barb adapter) includes a channel 50a and a varied cross section, generally depicted as reference numeral 52. In more specificity, the varied cross section includes a plurality of outward projections or barbs 52a, 52b, 52c and 52d, extending circumferentially about the body 52. The barbs 52a, 52b, 52c and 52d each include a ledge or shoulder 52a₁, 52b₁, 52c₁ and 52d₁ (hereinafter referred to as a shoulder) which is configured to prevent disengagement of the corrugated diaphragm from the barb adapter, amongst other features. In the embodiment of FIG. 10, the shoulders 52a₁ and 52b₁ are configured in the same orientation (e.g., facing in a same direction) and the shoulders 52c₁ and 52d₁ are configured in the same orientation (e.g., facing in a same direction). In further embodiments, the shoulders 52a₁ and 52b₁ are configured in a different orientation than that of the shoulders 52c₁ and 52d₁, e.g., the shoulders 52a₁ and 52b₁ face the shoulders 52c₁ and 52d₁.

The barbs 52a, 52b, 52c and 52d are configured and designed to couple the corrugated diaphragm 30 to the manifold 38, as well as ensure that the barb adapter 50 remains securely coupled to the manifold 38. By way of example, in embodiments, a major diameter of at least one of the barbs 52c and 52d, and preferably both of the barbs 52c and 52d, is larger than the inner diameter of the port 44, thus ensuring that the barb adapter 50 remains securely coupled to the manifold 38. In implementation, leakage is minimized between the major diameter of the barb adaptor 50 and the inner diameter of diaphragm neck. Such a configuration (orientation and size) also minimized leakage between the barb adapter 50 and the manifold 38 at designed pressures used in traction devices, i.e., at a pressure which are known to those of ordinary skill in the art. For example, a cervical traction device may be designed for pressures between 0 and 35 PSI; whereas, a lumbar traction device may be designed for pressure between 0 and 75 PSI. It should further be realized by those of skill in the art that the orientation of the barbs 52c and 52d ensures that the barb adapter cannot be easily removed from the manifold.

In further embodiments, the manifold 38 is made of a resilient type material, capable of slight deflection. In one preferred embodiment, the material of the manifold is softer than that of the barb adapter 50. For example, the material of the manifold 38 may include thermoplastic polyester elastomer; whereas, the material of the barb adapter 50 may be brass or other alloys or plastic materials, This minimizes air leakage between the barb adapter 50 and the manifold 38. The corrugated diaphragm 30 may also be of a softer material than the barb adapter 50.

Moreover, in embodiments, the combination of the major diameter (OD) of the barbs 52a and 52b and the thickness of the material of the neck of the corrugated diaphragm is larger than the inner diameter of the port 44, Thus, leakage is minimized between the major diameter of the barb adaptor and the inner diameter of the neck of the diaphragm and more specifically a barb feature on the barb adapter. It should further be recognized that the orientation of the barbs 52a, 52b, 52c and 52d facilitates installation of the barb adapter 50 into the neck 44, as well as the neck 30a of the corrugated diaphragm 30 about the barb adapter 50. Likewise, the orientation of the barbs 52a, 52b, 52c and 52d prevents the removal of the barb adapter 50 from the neck 44, as well as the neck 30a of the corrugated diaphragm 30 from the barb adapter 50.

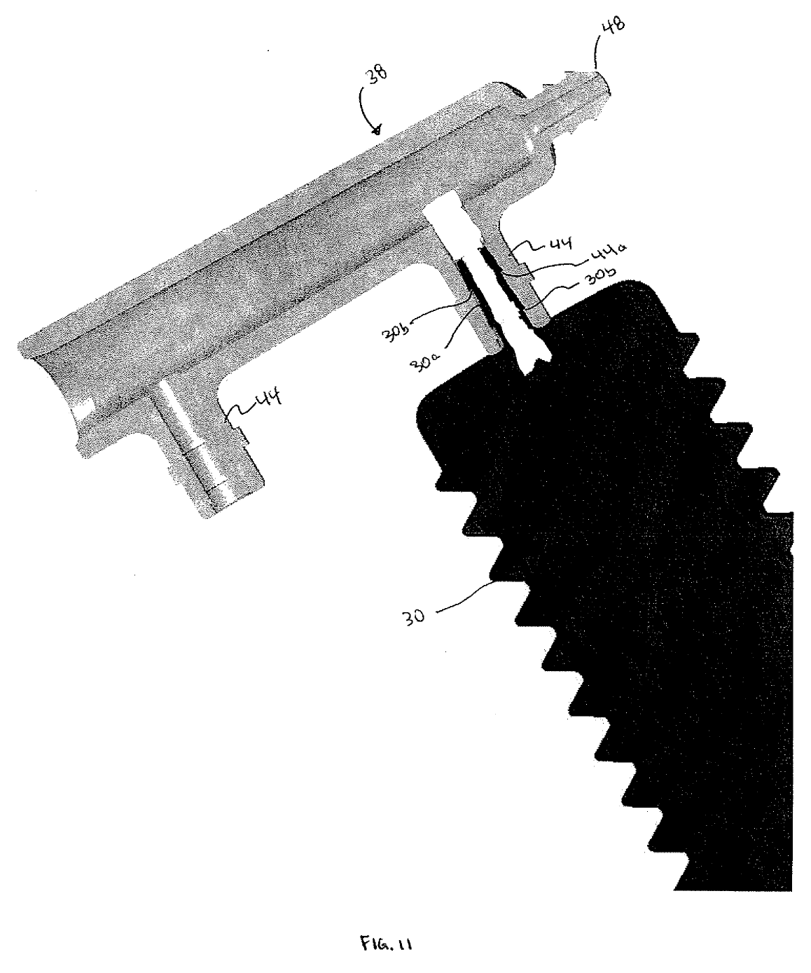

FIG. 11 shows another embodiment in accordance with invention. In this embodiment, the port 44 includes detents or grooves 44a about the entire circumference or portion thereof. In addition, the neck 30a of the corrugated diaphragm includes barbs 30b. The combination of the grooves 44a and barbs 30a are designed to secure the corrugated diaphragm 30 to the manifold 38 during curing of a glue or epoxy which is applied to secure the corrugated diaphragm 30 to the port 44. For example, in one embodiment, in the assembled state, the barbs 30b mate with the one or more detents 44a to stabilize the assembly during the curing stage. The corrugated diaphragm 30 may also be secured to the inner wall of the port 44 by way of ultrasonic or thermal welding. It should be recognized that the adapter barb 50 may also be used in combination with the embodiment of FIG. 11.

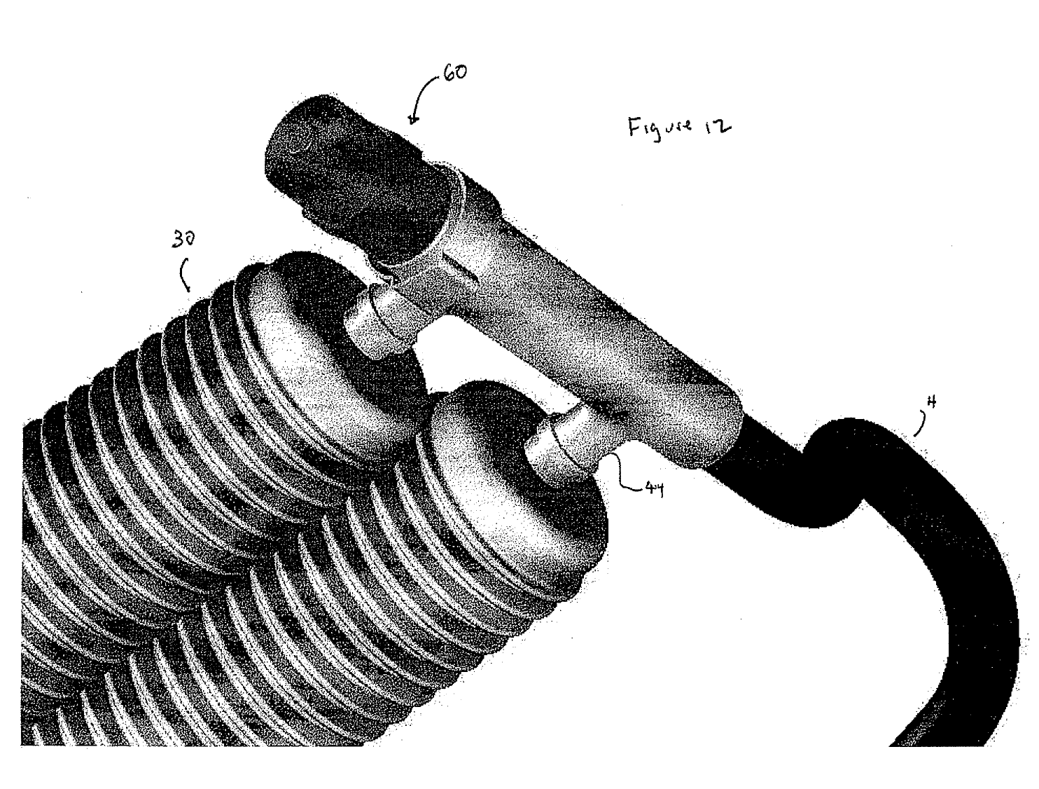

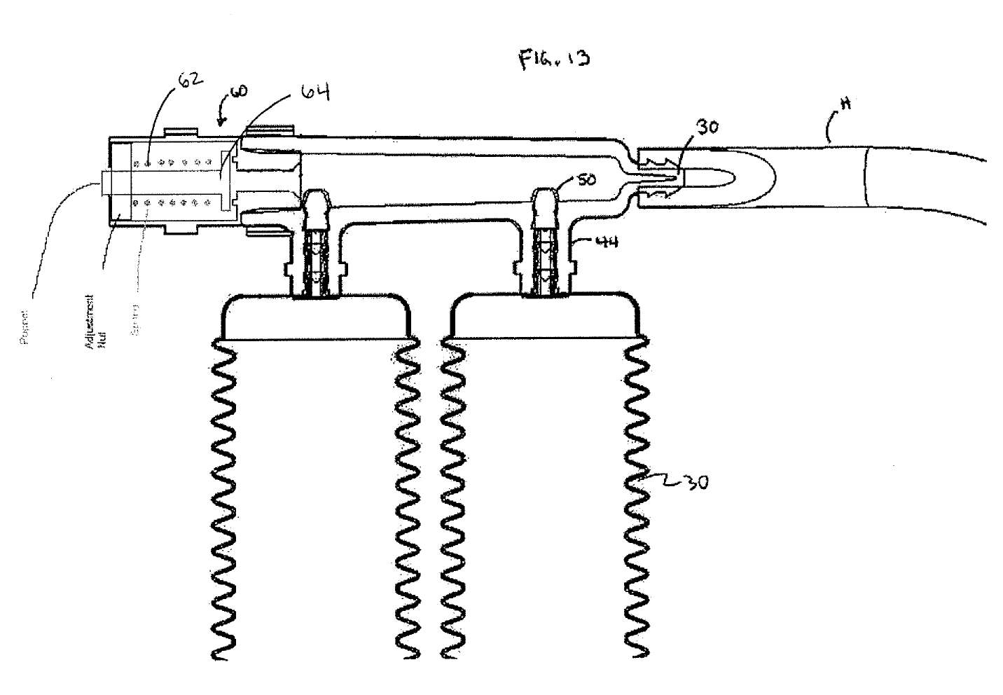

FIG. 12 shows a manifold with pressure release valve in accordance with the invention and FIG. 13 shows a cross section of the manifold along line 12-12 of FIG. 12. The pressure release valve is generally denoted as reference numeral 60 and includes a spring 62 that keeps a valve 64 closed under normal operating pressure. When excessive pressure occurs inside of the pneumatic system that exceeds the spring resistance, the valve 64 opens to allow the release of air until the pressure is reduced to within sate operating conditions. Thus, the pressure release valve ensures that a user cannot exceed a designed traction force, thus maintaining safe use conditions of the traction device. When the pressure reaches a value less than the spring resistance, the valve automatically closes.

### Operation of the Traction Device

To use the traction devices, the corrugated diaphragm type device 30 is initially at atmospheric pressure. With a cervical traction device, prior to use, the patient or user can adjust the angle of the cervical traction device by adjusting the positioning of the stand. The back of the user is placed on a support surface so that the wedges cradle the patient's neck. The wedges can also be adjusted but preferably remain substantially stationary during use of the device. The restraining device may be used about the patient's head to ensure the user remains substantially stationary during treatment of cervical traction device. In the lumbar device, the patient would strap the restraining device about his/her mid section.

Once properly positioned, the patient then pumps air into the corrugated diaphragm type device by way of the pneumatic circuit described above. The patient increases the traction force by manually operating the pump or decreases the traction force by manually pressing the pressure relief mechanism. As air is pumped into the corrugated diaphragm type device, the corrugated diaphragm type device will expand and move the carriage to apply a traction force. The entire structure of the cervical traction device, though, remains constant, during the application of the traction force.

When excessive pressure occurs inside of the pneumatic system, the pressure release valve will allow the release of air until the pressure is reduced to within safe operating conditions. Thus, the pressure release valve ensures that a user cannot exceed a designed traction force, thus maintaining safe use conditions of the traction device. When the pressure reaches a value less than the spring resistance, the valve automatically closes.

While the invention has been described in terms of embodiments, those skilled in the art will recognize that the invention can be practiced with modification.

What is claimed:

1. A device, comprising:
a frame;
a carriage slidable mounted on the frame;
at least one diaphragm connectable to the carriage, the at least one diaphragm is structured to elastically expand and retract, depending on an application of pressure, respectively, and to move the carriage relative to the frame; and
a manifold coupled between the at least one diaphragm and a pump system, the manifold comprising at least one port coupled to a neck of the at least one diaphragm and a neck communicating with the pump system.

2. The device of claim 1, wherein the at least one port is two ports extending along a length of the manifold.

3. The device of claim 2, wherein the two ports are coupled to two diaphragms.

4. The device of claim 1, wherein the neck is in fluid communication with the at least one port via a hollow body,

5. The device of claim 1, wherein the at least one diaphragm is at least one corrugated diaphragm.

6. The device of claim 1, wherein the neck includes one or more outward extending barbs configured to secure a hose of the pump system to the neck, wherein an end barb of the one or more outward extending barbs includes a tapered end to facilitate placement of a hose over the neck.

7. The device of claim 6, wherein an outermost diameter of at least one of the outward extending barbs is slightly larger than an inner circumference of the hose to minimize leakage between the hose and the manifold.

8. The device of claim 1, wherein the at least one diaphragm is secured to the at least one port via a glueless mechanism.

9. The device of claim 8, wherein the glueless mechanism is an adapter securing the at least one diaphragm with the at least one port to minimize leakage.

10. The device of claim 9, wherein the adapter includes a varied cross section which is configured to minimize leakage between the at least one diaphragm and the at least one port.

11. The device of claim 10, wherein the varied cross section includes a plurality of barbs extending circumferentially about the body, each of the barbs is configured to prevent disengagement of the at least one diaphragm from the adapter and the adapter from the manifold.

12. The device of claim 9, wherein the adapter is configured to be insertable into the at least one port and the at least one diaphragm.

13. The device of claim 9, wherein the manifold is a resilient type material softer than that of the adapter.

14. The device of claim 9, wherein a combination of a major diameter (OD) of a portion of the adapter and thickness of material of a portion of the at least one diaphragm is larger than an inner diameter of the at least one port.

15. The device of claim 1, wherein the at least one port includes at least one detent about a circumference or portion thereof and a neck of the at least one diaphragm includes at least one corresponding barb.

16. The device of claim 15, wherein the at least one detent and corresponding barb is configured to secure the at least one diaphragm to the manifold during curing of a glue or epoxy applied between the at least one diaphragm and the at least one port.

17. The device of claim 15, wherein the at least one diaphragm and the at least one port are ultrasonic or thermal welded together.

18. The device of claim 1, wherein the manifold includes a pressure release valve.

19. The device of claim 8, wherein the manifold includes a pressure release valve.

20. The device according to claim 1, wherein the at least one diaphragm is corrugated and has a spring force such that the at least one corrugated diaphragm retracts and moves the carriage toward an initial position upon the release of pressure.

21. The device according to claim 1, wherein the device is a cervical traction device or a lumbar traction system.

22. The device according to claim 1, further comprising a wedge system that is only linearly adjustable relative to the carriage.

23. The device according to claim 1, further comprising:
a stand slidably mounted to the frame, the stand providing different adjustable angles for the frame relative to a supporting surface; and
a locking system which locks the stand at a plurality of different locations on the frame, the locking system including a spring arm or a hook, and notches or holes associated with a guide on the frame which is structured to accommodate the spring arm or the hook.

24. The device according to claim 22, wherein each wedge of the wedge system is adjustable by a pinch spring arm mechanism and includes an anti-rotation device to prevent rotation of wedges of the wedge system.

25. A device comprising a frame for accommodating at least one corrugated diaphragm device, the at least one corrugated diaphragm device having a retractable spring force such that, upon release of pressure within the at least one corrugated diaphragm device, the at least one corrugated diaphragm device retracts and moves the carriage towards an initial position; and a manifold connectable between the least one corrugated diaphragm device and a pneumatic system, the manifold including at least one port and at least one neck, the at least one port connectable to a neck of the least one corrugated diaphragm device and the at least one neck connectable to a hose of the pneumatic system.

26. The device of claim 25, wherein the neck includes one or more outward extending barbs configured to secure a hose of the pump system to the neck, an end barb of the one or more outward extending barbs includes a tapered end to facilitate the placement of the hose over the neck and an outermost diameter of at least one of the outward extending barbs is slightly larger than an inner circumference of the hose to minimize leakage between the hose and the manifold.

27. The device of claim 25, further comprising an adapter configured to secure the at least one corrugated diaphragm device with the at least one port, wherein the adapter includes a varied cross section which is configured to provide minimize leakage between the at least one corrugated diaphragm device and the at least one port.

28. The device of claim 27, wherein the varied cross section includes a plurality of barbs extending circumferentially about a body, each of the barbs is configured to prevent disengagement of the at least one corrugated diaphragm device from the adapter and the adapter from the manifold.

29. The device of claim 27, wherein the adapter is configured to be insertable into the at least one port and the at least one corrugated diaphragm device.

30. The device of claim 27, wherein the manifold is a resilient type material softer than that of the adapter.

31. The device of claim 25, wherein the at least one port includes at least one detent about a circumference or portion thereof and a neck of the at least one corrugated diaphragm includes at least one corresponding barb.

32. The device of claim 25, wherein the at least one detent and corresponding barb is configured to secure the at least one corrugated diaphragm to the manifold.

33. The device of claim 25, wherein the at least one corrugated diaphragm and the at least one port are glued, ultrasonic or thermal welded together.

34. The device of claim 25, wherein the manifold includes a pressure release valve.

35. The device according to claim 25, further comprising a wedge system that is only linearly adjustable relative to the carriage.

36. A device comprising:
a body having at least one port and one neck having a channel therein in fluid communication with the at least one port, the neck include a projection, and
an adapter configured to be insertabe into the at least one port, the adapter comprising a varied cross section with a major diameter of at least one portion of the varied cross section being larger than an inner diameter of the at least one port at least in one location.

37. The device of claim 36, wherein the at least one port is two ports extending along a length of the manifold.

38. The device of claim 36, wherein the neck includes one or more outward extending barbs configured to secure a hose thereto, wherein an end barb of the one or more outward extending barbs includes a tapered end.

39. The device of claim 36, wherein the varied cross section includes a plurality of barbs extending circumferentially about the body, each of the barbs is configured to prevent disengagement of a diaphragm from the adapter and the adapter from the manifold.

40. The device of claim 36, wherein the manifold is a resilient type material softer than that of the adapter.

41. The device of claim 36, wherein the at least one port includes at least one detent about a circumference or portion thereof.

42. The device of claim 36, wherein the manifold includes pressure release valve.

43. The device of claim 36, further comprising a traction device comprising:
a frame;
a carriage slidable mounted on the frame; and
at least one diaphragm connectable to the carriage, the at least one diaphragm of pressure, respectively, and to move the carriage relative to the frame,
wherein the at least one port is secured with the at least one diaphragm, and the adapter is configured to be insertable into the at least one port and a neck of the at least one diaphragm.
